(19) United States
(12) Patent Application Publication
(10) Pub. No.: US 2009/0093864 A1
(43) Pub. Date: Apr. 9, 2009

(54) METHODS AND DEVICES FOR APPLYING ENERGY TO TISSUE

(76) Inventor: Robert S. Anderson, Livermore, CA (US)

Correspondence Address: BLAKELY SOKOLOFF TAYLOR & ZAFMAN LLP, 1279 OAKMEAD PARKWAY, SUNNYVALE, CA, 94085-4040, US

(21) Appl. No.: 11/868,833

(22) Filed: Oct. 8, 2007

(51) Int. Cl. A61N 5/06; A61F 7/00

(52) U.S. Cl. 607/88; 607/99

(57) ABSTRACT

Methods, systems, and devices to treat a region of skin; the treatment may be used to stimulate the production of collagen or destroy adipose tissue. The region of skin is exposed to a uniform energy application or series of applications. The region of skin may be exposed to positive and negative pressures. Therapeutic substances may be applied to the region of skin.

### FIELD OF THE INVENTION

The present invention relates to methods, devices, and systems treating skin, and in certain embodiments the invention relates to methods, devices, and systems for stimulating the production of collagen in the skin.

### BACKGROUND

A primary component of the human skin is collagen, which is a fibrous protein that is secreted by fibroblast cells. Collagen exists in an extracellular matrix (ECM) which is part of the dermis of the human skin. There are several types of collagen, of which Type-I and Type-III collagen being predominant in the skin. The ECM is a meshwork of long collagen helical structures, as well as other macromolecules. The ECM attaches to cells using proteins called integrins. Integrins are also responsible for cell signaling.

New collagen will be formed by fibroblast cells when an injury occurs to the ECM. Devices have been created to purposely injure the ECM in order to produce new collagen. Examples of which are found in US patent applications US 2005-0251118 A1, US 2006-0189964 A1, and US 2005-0251117A1. Many of the devices in some part use laser light, ultrasound, and radio frequency (RF) energy sources. Heating skin above 65° C. will denature the collagen and cause new growth. Recently devices using energy sources heat collagen to a temperature where the collagen molecules are fractured. The fractured collagen molecules generate cytokines which trigger a growth factor that stimulates fibroblasts to generate new collagen. This procedure is used to reduce wrinkles and generally smooth the appearance of skin.

In addition to modifying and generating new collagen, RF devices are being used to destroy adipose tissue. The adipose cells are heated by the RF energy to a temperature high enough to destroy the cell walls. The triglycerides and other substances escape from the interior of the cell and are removed or redistributed by the body's natural systems.

Many systems have encountered problems with the non uniformity of distributed energy. Typically past systems apply energy through a flat electrode surface with the intended goal of uniform distribution of the energy in the tissue. Non uniformities in the tissue tend to increase the RF energy is some areas and decrease it in others. One of the non uniformities is the blood supply; blood is highly conductive while the surrounding tissue may be poorly conductive. The RF energy will concentrate in the blood system and not properly heat the tissue, of which the result is localized burns surrounded with sections of untreated tissue.

### SUMMARY OF THE DESCRIPTION

Methods, systems, and devices to treat a portion of skin and/or other biological tissue are described. The invention provides, in one embodiment, a medical device for treating a portion of skin, including a housing including a outer housing surface, an inner housing surface at least partially defining a main chamber for treating a volume of skin, and a sealing surface, wherein placing the sealing surface against a portion of skin forms a portion of the main chamber, the housing surface including at least one channel in fluid communication with a pressure source, and a needle electrode coupled to the inner housing surface, wherein the electrode is recessed within the main chamber and configured to apply energy when negative pressure is applied through at least one channel and when the needle pierces the portion of skin.

The invention also provides, in one embodiment, a method for treating a portion of skin, including applying a device against a portion of skin, wherein the device includes a needle located in a chamber, the needle configured to apply energy to the volume of skin, wherein the needle is not in contact with the portion of skin when the device is applied, applying negative pressure to the portion of skin with the device to bring a volume of skin into the chamber, piercing the volume of skin with the needle, and activating the device to apply an energy to the volume of skin. The activating may be performed automatically in response to the contacting of the skin by the needle; this may occur automatically by sensing the contact electrically and then activating in response to the sensing.

The invention also provides, in one embodiment, a medical device for treating a portion of skin, including a housing including a housing surface for mating against a portion of skin, and a plurality of electrodes coupled to the housing surface, wherein each electrode activates incrementally over time with respect to each other electrode or sets of electrodes, and wherein each electrode activates, in one embodiment, only when in sufficient contact with the portion of skin.

The invention also provides, in one embodiment, a medical device for treating a portion of skin, including a housing including a outer surface, an inner surface defining a recessed chamber, and a sealing surface for sealing the inner surface against a portion of skin, an optional spacer coupled to the inner surface and within the recessed chamber, the spacer including a plurality of spacer passages which communicate with the inner surface, and wherein at least one passage is in fluid communication with a pressure source, a plurality of electrodes coupled to the spacer, each electrode located within a spacer passage, and a return electrode plate coupled to the spacer, the return electrode plate including a plurality of plate passages corresponding to spacer passages.

The invention also provides, in one embodiment, a medical device component for treating a portion of skin, including a housing including a outer surface an inner surface defining a recessed chamber, and a mating surface for sealing the inner surface against a portion of skin, and a connecting surface for connecting to a second device, an optional spacer coupled to the inner surface and within the recessed chamber, the spacer including a plurality of spacer passages which communicate with the mating surface, and wherein at least one passage is in fluid communication with the connecting surface, a plurality of treatment electrodes coupled to the spacer, each treatment electrode located within a spacer passage, a return electrode coupled to the housing, and an electrical connector coupled to the housing, the electrical connector electrically connected to the treatment electrodes and return electrode.

The invention also provides, in one embodiment, a method for treating a portion of skin, including applying a device against a portion of skin, wherein the device defines a volume of skin for treatment, activating the device to apply a plurality of energy applications to the volume of skin, wherein the plurality of energy applications are applied by the device incrementally over time through different sets of electrodes, such as needle electrodes which contact and may pierce the skin, during a single application of the device, and wherein each respective energy application is applied to a different portion of the volume of skin.

The invention also provides, in one embodiment, a method for treating a portion of skin, including triggering a device to apply negative pressure to a portion of skin, the device having a plurality of electrodes which are contact with portion of skin when the negative pressure is applied, testing the electrodes for impedance values, wherein the portion of skin is included in a circuit between the electrodes an a return electrode, determining if the impedance values are appropriate to allow an application of therapeutic energy, and applying therapeutic energy to the device, wherein the therapeutic energy is transferred to the portion of skin by the plurality of electrodes, and wherein the therapeutic energy is applied sequentially to each electrode.

The invention also provides, in one embodiment, a medical device component for treating a portion of skin, including a controller for supplying energy to a hand held device, a hand held device coupled to the controller; and a tip detachably coupled to the hand held device, the tip including a housing including a outer surface, an inner surface defining a recessed chamber, and a mating surface for sealing the inner surface against a portion of skin, and a connecting surface for connecting to a second device, an optional spacer coupled to the inner surface and within the recessed chamber, the spacer including a plurality of spacer passages which communicate with the mating surface, and wherein at least one passage is in fluid communication with the connecting surface, a plurality of treatment electrodes coupled to the spacer, each treatment electrode located within a spacer passage, a return electrode coupled to the housing, and an electrical connector coupled to the housing, the electrical connector electrically connected to the treatment electrodes and return electrode.

The invention also provides, in one embodiment, a system for treating a portion of skin including, a controller for supplying energy to a hand held device, a hand held device coupled to the controller, and a tip detachably coupled to the hand held device, the tip including a housing including a outer surface, an inner surface defining a recessed chamber, and a mating surface for sealing the inner surface against a portion of skin, and a connecting surface for connecting to a second device, an optional spacer coupled to the inner surface and within the recessed chamber, the spacer including a plurality of spacer passages which communicate with the mating surface, and wherein at least one passage is in fluid communication with the connecting surface, a plurality of treatment electrodes coupled to the spacer, each treatment electrode located within a spacer passage, a return electrode coupled to the housing; and an electrical connector coupled to the housing, the electrical connector electrically connected to the treatment electrodes and return electrode.

### DESCRIPTION OF THE DRAWINGS

The present invention is illustrated by way of example and not limitation in the figures of the accompanying drawings in which like references indicate similar elements.

FIG. 1 is a layout describing a system for treating a portion of skin.

FIG. 2 perspective view of a device for treating a portion of skin.

FIG. 3a is a cross sectional view of a device for treating a portion of skin.

FIGS. 3b and 3c are bottom views of devices for treating a portion of skin.

FIG. 4a is a cross sectional view of a device for treating a portion of skin.

FIGS. 4b-4h are a cross sectional detail views of devices for treating a portion of skin.

FIGS. 5a-5d are cross sectional views of a device on a portion of skin showing a method for treating the portion of skin.

FIG. 6 is a flow chart for a method for treating a portion of skin.

FIG. 7 shows a cross section of a portion of a device for treating a portion of skin.

### DETAILED DESCRIPTION

Various embodiments and aspects of the inventions will be described with reference to details discussed below, and the accompanying drawings will illustrate the various embodiments. The following description and drawings are illustrative of the invention and are not to be construed as limiting the invention. Numerous specific details are described to provide a through understanding of various embodiments of the present invention. However, in certain instances, well-known or conventional details are not described in order to provide a concise discussion of embodiments of the present inventions.

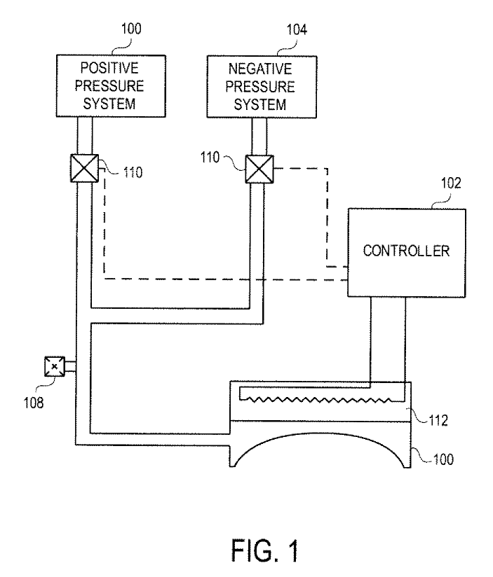

FIG. 1 shows a system for treating a portion of skin, according to an embodiment of the invention. The system includes a device 100 which can apply positive pressure (e.g. pressures slightly above normal atmospheric or higher pressures) and negative pressure (e.g. pressures below atmospheric pressure such as a partial vacuum) to a portion of skin on a patient, and the device 100 can include a controller 102, a positive pressure source 104, and a negative pressure source 106. The controller 102 regulates the application of pressure by monitoring a pressure sensor 108 and electronically controlled regulator mechanisms 110 which may be valves which are opened and closed under control of controller 102. The device forms a sealed internal volume when placed against the skin of a patient. The device 100 may include a set of RF electrodes 112 which may be, in one embodiment, one or more separate needle electrodes controlled by the controller 102. The controller 102 supplies the device 100 with RF energy which may be delivered through the one or more needle electrodes.

Positive and negative pressures are applied at the device 100 in sequential turns electronically controlled by the controller 102. Prior application number US 2005/0251117 A1 describes examples of positive/negative/positive sequences which may be generated by device 100 when used in various embodiments described herein. The positive pressure required is pressure above atmospheric pressure large enough to detect a good seal against the skin while at the same time not forcing the device off the patient, approximately 1-3 psi or 7−21 kPa above atmospheric pressure. The negative pressure required may be pressure below atmospheric pressure enough pressure to draw a volume of skin into the device and affect the fibroblast cells and ECM, approximately 3 psi or 20 kPa below atmospheric pressure. A volume of skin may be drawn in the device for as little as a few seconds or less (e.g. 0.05 seconds) to as long as an hour.

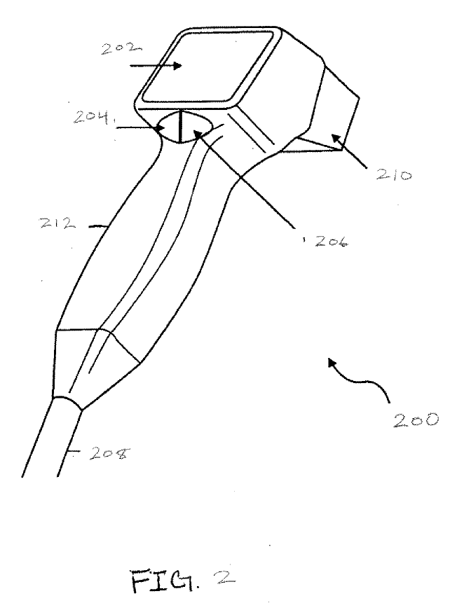

FIG. 2 is a perspective view of a handheld device 200 to apply uniform energy to biological tissue. It is appreciated that device 200 may include one or more features described herein, e.g., substance suppliers, energy sources, pressure conduits, articles to push blood out of the treatment area, and sensors and device 100 may be implemented in the form factor shown in FIG. 2 as device 200. As shown in FIG. 2, device 200 includes a pixilated display with multiple rows and columns of pixels that may display a status of the device (e.g., “Standby” or “On” or “Treating”), a energy source status (e.g., Low or Medium or High, along with a bar graph), a vacuum status (e.g., “Low” or “High”), the skin's temperature, the skin's color, and the patient's pulse count. In an embodiment, display 202 may be a liquid crystal display (“LCD”), or a light emitting diode (“LED”) display. Device 200 may have a power adjustment control 206 to control the amount of energy that is applied to the biological external tissue (e.g., to adjust the power level of RF energy). The device 200 also includes a pneumatic adjustment control 204 to control a level of a vacuum that is applied from a vacuum pump (not shown) to a chamber of the device. Device 200 may include a substance quantity adjustment control that may be used to regulate the amount of medicine, (e.g. an acne treatment solution) lotion, or other substance that is administrated in the procedure. Device 200 includes a cable 208 to provide power and pressures for device operation. For example, cable 208 may include one or more electrical wires to connect device 200 to an electrical outlet, and one or more conduits to provide negative and positive pressure to a chamber of device 200. As shown in FIG. 2, device 200 has a detachable tip 210, which contains a chamber, which is applied to a biological tissue. In one embodiment, tip 210 may be disposable. Cable 208 may provide electrical connection to a standalone central control station, such as controller 102. In one embodiment, the standalone central control station may be a computer that has a printer and/or storage device(s) for storing data provided by device 200. In one embodiment, disposable tip 210 on device 200 may be a disposable membrane and may be custom designed to fit a particular type and a size of a biological tissue. For example, disposable tip 210 may have a different size for large areas of a biological tissue versus small areas of biological tissue, and may be shaped differently in accordance with contours of a biological tissue. The handle 212 of device 200 may be designed to fit a particular size of hand and may have groves to fit a particular hand size in some embodiments.

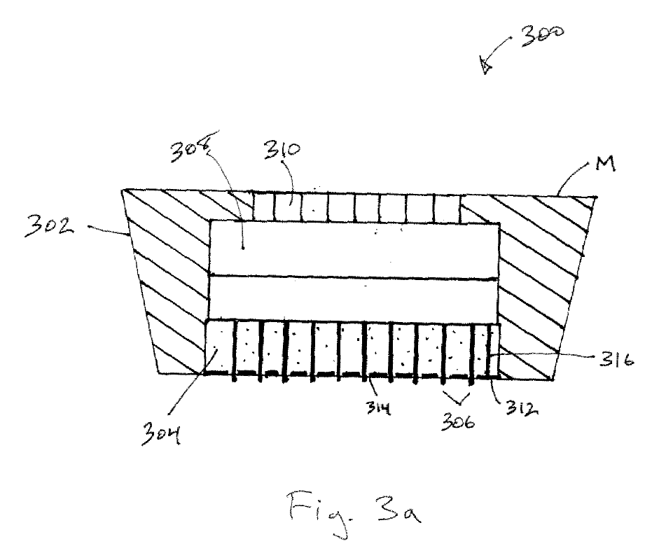

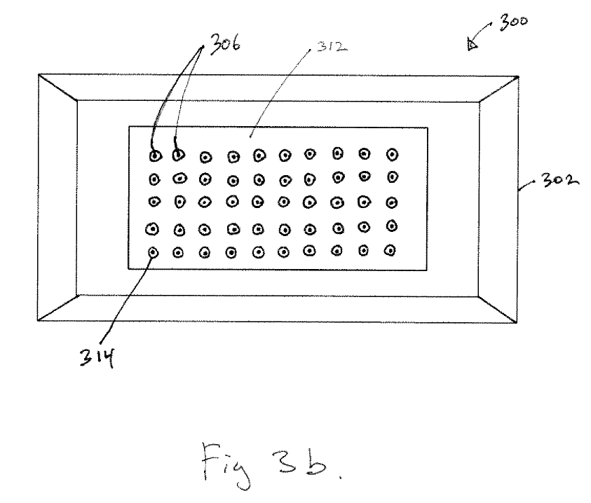

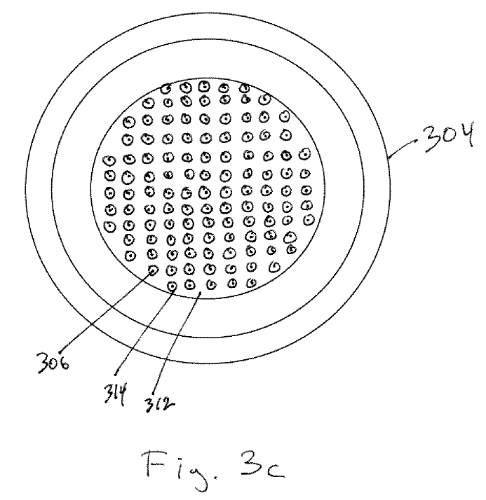

FIG. 3a shows a cross section of a device 300 for applying uniform energy to tissue. The device includes a housing 302. The housing is preferentially constructed from a material of low electrical conductance, for example a polymer or ceramic material. As shown the housing is intended to detachably couple as the tip of a hand held unit, for example what is shown in FIG. 2, which supplies necessary power connections to the device 300. Alternatively, the housing may be a stand alone unit, i.e. an ergonomic housing with the ability to operate independently with appropriate power connections.

The device 300 may be reusable or disposable after one or a plurality of uses. The device 300 may include a solid state circuitry (not shown) which counts the number of instances the device is used. The handheld unit, to which the device 300 detachably couples to, in turn would only supply power to a device 300 that has a predetermined amount of uses (e.g. zero uses, five uses, etc.). The housing 302 includes a surface M for mating to a secondary device (e.g. device 200). The surface M includes various connectors (not shown) for required resources such as power, pressure, cooling fluids, and therapeutic substances if necessary.

The device 300 includes, in one embodiment, a spacer 304. The spacer 304 serves to house a plurality of electrodes 306. The spacer 304 may be constructed from various compatible ceramics (e.g. silicon dioxide) or high temperature polymer (e.g. PTFE). In general the ceramic should be of low electrical conductance. The spacer 304 includes a plurality of channels or passages which house at least one electrode 306. Alternatively, the housing 302 may incorporate the function of the ceramic spacer 304, and thus the ceramic spacer would be unnecessary, thus the housing would be required to made from a material capable of withstanding high temperature, for example PTFE.

The device 300 may include a thermoelectric module 308. The thermoelectric module 308 is coupled to the spacer 304 preferably by a thermally conductive adhesive. Heat generated by the electrodes may be conducted from the ceramic spacer 304 and to a heat sink 310 located opposite to the ceramic spacer 304. The thermoelectric module 308 may also function to keep the ceramic spacer 304 at a temperature which is lower than an ambient temperature in order to provide a more comfortable surgical procedure.

The device 300 includes electrodes 306 which are coupled to the spacer 304. The electrodes 306 transfer RF energy to tissue which comes into contact with the electrodes 306. The electrodes 306 may be configured as needles (hollow or solid) or blunt probes, and thus respectively the electrodes 306 may pierce or deform skin. The electrodes 306 are configured to extend past the housing 302 or alternatively flush or within the housing. If the electrodes 306 are flush with or within the housing, then negative pressure channels may be incorporated into each electrode passage in order to draw tissue into the electrode. Each electrode 306 includes a conductor (not shown) in electrical communication with a connector (not shown) mounted to the housing for connection with the hand held device 200 and each electrode may be coupled to a controller, such as controller 102, which regulates how and when energy is applied to one or more electrodes (e.g., sequentially and incrementally). The electrodes 306 are configured to activate independently from each other or incrementally with respect to each other in one embodiment. The number of electrodes in the device 300 may be as few as one, and there is no limit to the total number of electrodes that the device may include.

Independent or incremental activation is particularly advantageous in order to provide a uniform treatment to the skin, which thereby provides a uniform treatment and avoids localized burns and untreated portions of tissue. By independent activation it is meant that each electrode would be supplied RF energy independently from other electrodes, and thus each electrode would have a power source which applies load dependent RF energy. Load will vary per electrode depending on impedance factors which include electrode contact, tissue type, and blood vessel interaction. For example an electrode in close vicinity to a blood vessel will have a lower load than an electrode which is not in the same vicinity, and thus if the electrodes shared the same power source more RF energy will flow to the electrode near the blood vessel and the electrode with the higher load may provide in sufficient treatment.

Incremental activation is an alternative to independent activation. Incremental activation can use the same power source for all electrodes, however each set of electrodes is switched such that only one electrode (or set of electrodes) is activated (e.g. RF energy is applied) at a given time. Incremental activation ensures that each electrode (or set of electrodes) will receive enough power regardless of individual electrode load conditions. The time between activations may be very short (e.g. milliseconds) so that overall procedure time would not be significantly effected. A complete cycle of electrode activations may also be repeated as necessary in order heat a given portion of tissue to a desired temperature, Incremental activation may be controlled by a controller, such as controller 102, which is coupled to the electrodes. The electrodes may be separated into groups or sets which are activated separately and incrementally over time.

The electrodes 306 may be configured to sense tissue contact before activation. On method of sensing tissue involves supplying very low RF power to each electrode in order to determine the proper impedance value upon tissue contact. Once a low enough impedance value is detected, either by the hand held device 200 or the controller 102, the electrode would be supplied with full RF power to effectuate treatment. The hand held device 200 or controller 102 may be equipped with indicators (e.g. readouts, lights, audible warnings) to signal impedance values which are too high, and thus instruct an operator to reposition or change treatment areas.

The device 300 includes a return electrode plate 312 coupled to the spacer 304. The return plate 312 serves, in one embodiment, as a return path for RF energy passed through the electrodes 306 and subsequently through tissue, thus the device as shown is a bipolar configuration. The return plate 312 may be coupled to the ceramic spacer 304 by use of an adhesive or through mechanical securing (e.g. screws, snap fit, etc.). The return plate includes holes 314 which align and match the electrode channels. The return plate holes 314 may be slightly larger or equivalent in size as compared to the electrode channels. The return plate 314 includes a conductor 316 which is in turn electrically connected to the hand held device 200. The return plate may be constructed from a variety of conductive metals (e.g. stainless steel, gold, etc.).

Alternatively a mono-polar configuration may be implemented and the return plate 312 would not be used. Alternatively other electrodes 306 may be switched to be return electrodes when used in an incremental activation mode as described above and the return plate 312 would not be necessary. Preferably at least two electrodes 306 would be switched to become return electrodes for every one active electrode 306, but only one electrode 306 switched to become a return electrode per every active RF electrode is necessary. In use, this process would be repeated for every electrode.

FIG. 3b shows the bottom view or the treatment side of the device 300. The electrodes 306 are patterned in a grid formation in the ceramic spacer 304. The device 300 is not limited to a grid formation, and other patterns for example circular. More or less electrodes than shown may also be used. FIG. 3c shows an alternative bottom view of the treatment side of the device 300. The alternative construction features a circular shaped housing.

In use, the device 300 is applied to a portion of skin for application of treatment. Suction may then applied through the electrode channels, if the device has recessed or flush electrodes, to bring tissue into direct contact with each respective electrode. The device 300 then may detect impedance values for each respective electrode. If the impedance values are within an acceptable range, then each respective electrode may activate with RF energy, either independently or incrementally as described above. The process may be repeated until the portion of skin is heated to a satisfactory level to provide the desired therapy (e.g. collagen generation). Negative pressure is ceased when the therapy is completed, and positive pressure may be used to forcibly release the portion of skin.

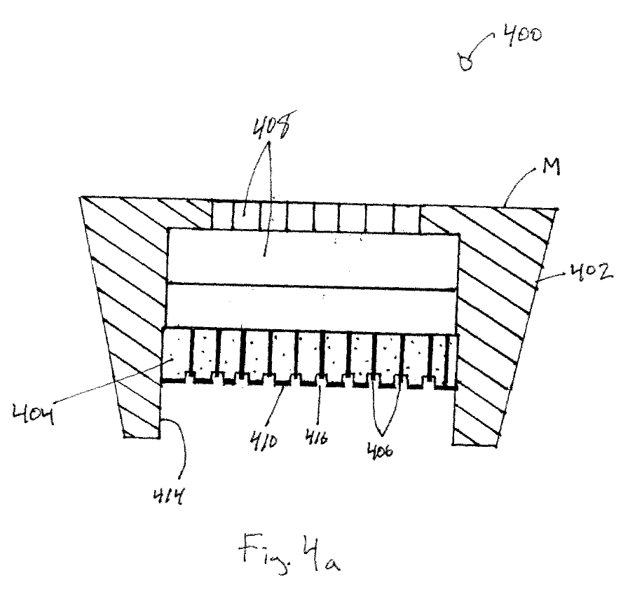

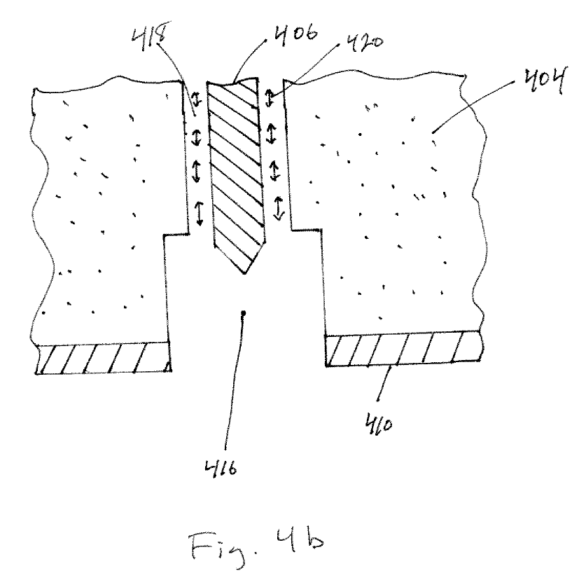

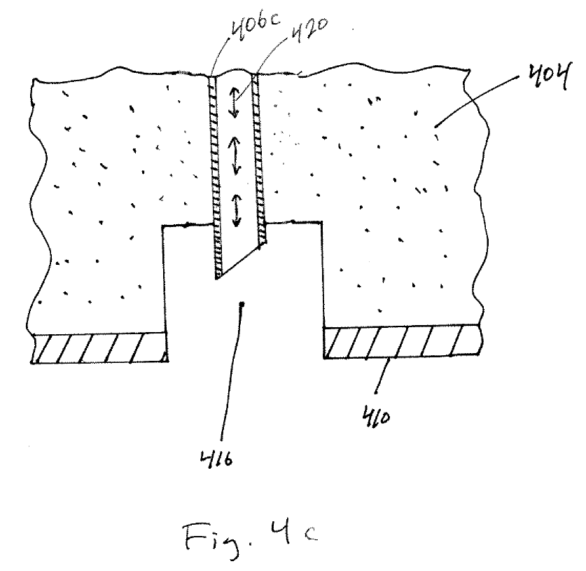

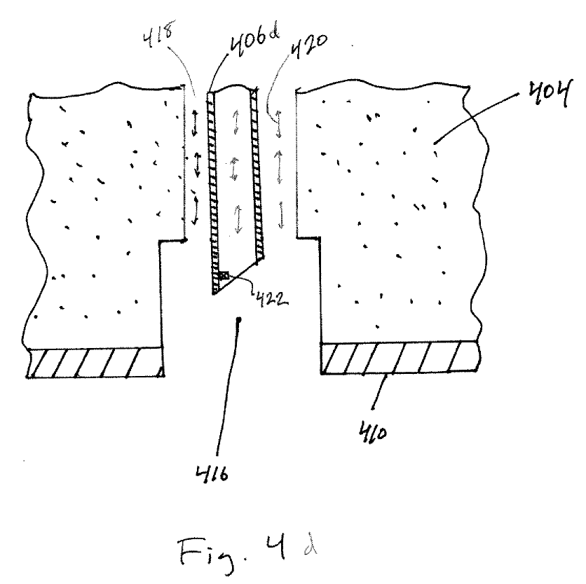

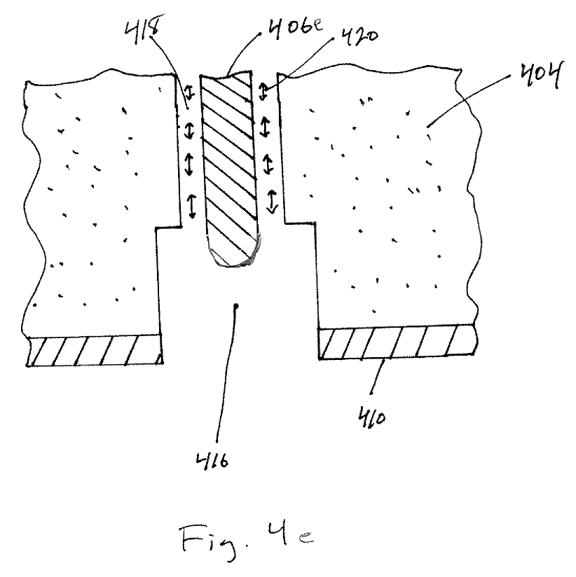

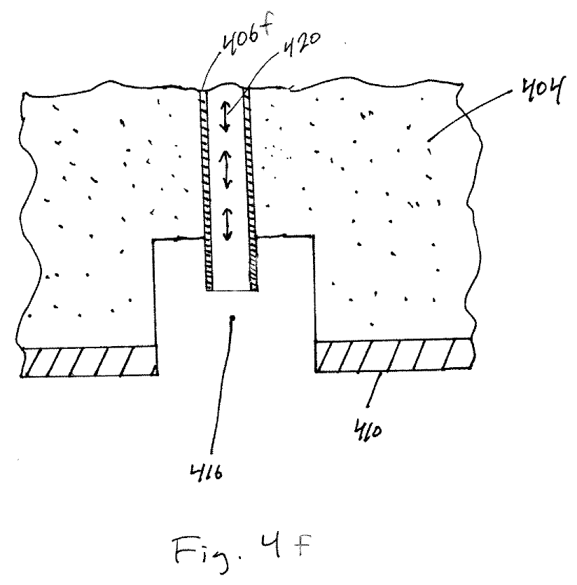

FIG. 4a shows a cross section of a device 400 for applying energy to tissue. The device 400 is an alternate construction of device 300 in FIG. 3a. The device 400 may include a housing 402, a ceramic spacer 404, electrodes 406, a thermoelectric cooler assembly 408, and a return plate 410. The housing 402 includes a surface 412 which defines a main chamber 414. The housing 402 may, in one embodiment, be a removable tip which is designed to attach to the operating end of device 200. The return plate 410 and ceramic spacer 404 form the upper portion of the main chamber 414. The housing 402 includes a surface M for mating to a secondary device (e.g. device 200). The surface M includes various connectors (not shown) for required resources such as power, pressure cooling fluids, and therapeutic substances if necessary. The electrodes 406 are coupled to a controller which, in one embodiment, applies RF energy to the electrodes in a controlled manner. This controller manner may include, for example, applying the RF energy in an incremental activation manner such that different sets of electrodes are activated sequentially over time. For example, a first set of electrodes in functional content with a first area of the skin may receive RE energy during a first time period (while a second set of electrodes does not receive the RF energy during the first time period, and during a second time period (e.g., a time period immediately subsequent to the first time period), the second set of electrodes, which are in functional contact with a second area of the skin, receive RF energy while the first set does not receive RE energy. This activation may be repeated for additional sets of electrodes in order to sequentially apply RF energy to the different sets of electrodes; it will be appreciated that a set may be a single electrode or multiple electrodes. In the case of a single electrode being a set, a common return path through a surface (e.g. return plate 410) may be used. In the case of multiple electrodes being a set, one electrode in the set may be one path and another electrode in the set may be a return path and no common return path is needed.

The ceramic spacer 404 includes a plurality of electrode chambers 416 in which the electrodes 406 are coupled to. The electrode chambers 416 are fluidly connected to a pressure source via the hand held device 200, which may provide both positive and negative pressure to a portion of skin. The main chamber 414 and electrode chambers 416 use negative pressures to draw in the portion of skin for treatment, and more precisely define a volume of skin for treatment. The ability to precisely define a volume of skin for treatment makes device 400 preferable to device 300 for non-planar portions of skin. The main chamber 414 may also be provided with secondary channels for providing positive and negative pressure to the portion of tissue.

FIG. 4b shows a close up cross section of an electrode chamber 416. The chamber is defined by several surfaces, including the ceramic spacer 404 and the return plate 410. The electrode 406 is shaped as a needle which may pierce the skin, alternatively the electrode may be blunt and not pierce the skin. The electrode 406 is located within a pressure channel 418 defined by the ceramic spacer 404. Arrows 420 indicate the direction of pressure flow, which may be either positive or negative air pressure. Alternatively therapeutic substances in a liquid form may applied through the pressure channel 418, such as acne medication (e.g. benzoyl peroxide) or lotion, and heated by the electrodes to lower the viscosity of the therapeutic substances and thereby enable the therapeutic substances to flow deeper into the skin and desired treatment areas. Therapeutic substances may be applied prior to, during, or subsequent to the application of energy. Some pressure channels 418 may be devoted exclusively for the application of positive and negative pressure, and some channels may be devoted exclusively for the application of therapeutic substances. Alternatively a cooling fluid may be applied through the pressure channel 418 prior and subsequently to an application of energy and/or each electrode may be coupled to a cooling device such as a thermoelectric cooler.

FIG. 4c shows an alternative close up cross section of an electrode chamber 416. The chamber 416 is as generally described in FIG. 4b. The electrode 406c is a hollow needle wherein positive and negative pressure 420 may be applied through the electrode. Alternatively therapeutic substances may be applied through the needle as described above. Alternatively a cooling fluid may be applied through the needle prior and subsequently to an application of energy and/or each electrode may be coupled to a cooling device such as a thermoelectric cooler.

FIG. 4d shows an alternative close up cross section of an electrode chamber 416. The chamber is as generally described in FIG. 4b. The electrode 406 is a hollow needle wherein therapeutic substances or positive and negative pressure 420 may be applied through the electrode and through the pressure channel 418. The electrode 406d also includes an optional temperature sensing device 422 (e.g. thermocouple) attached to the distal portion of the needle to measure temperature of the tissue. The temperature sensing device 422 may be used in all of the embodiments disclosed herein.

FIG. 4e shows an alternative close up cross section of an electrode chamber 416. The chamber 416 is as generally described in FIG. 4b. The electrode 406e is a blunt probe wherein the electrode 406e does not pierce the skin, and positive and negative pressure 420 may be applied through the pressure channel 418. Alternatively therapeutic substances may be applied through the through the pressure channel 418 as described above. Alternatively a cooling fluid may be applied through the pressure channel 418 prior and subsequently to an application of energy.

FIG. 4f shows an alternative close up cross section of an electrode chamber 416. The chamber 416 is as generally described in FIG. 4b. The electrode 406f is a hollow blunt probe wherein the electrode 406f does not pierce the skin, and positive and negative pressure 420 may be applied through the electrode 406f. Alternatively therapeutic substances may be applied through the blunt probe as described above. Alternatively a cooling fluid may be applied through the probe prior and subsequently to an application of energy.

FIG. 4g shows an alternative close up cross section of an electrode chamber 416. The chamber is as generally described in FIG. 4b. The electrode 406g is a hollow blunt probe wherein the electrode 406g does not pierce the skin, and wherein therapeutic substances or positive and negative pressure 420 may be applied through the electrode and through the pressure channel 418. Alternatively a cooling fluid may be applied through the probe and through the pressure channel 418, prior and subsequently to an application of energy.

FIG. 4h shows alternative close up cross section of an electrode chamber 416. The chamber is as generally described in FIG. 4b. The electrode 406h is configured as a hollow needle. A light source (not shown) is coupled to the electrode 406h in order to provide therapeutic light 422 through the electrode 406h. The light source may provide incoherent light, coherent light, and/or alternatively non-visible light and/or electromagnetic radiation in the range of a radio frequency spectrum. The light source may be a flash lamp, arc lamp, laser, or an LED. A fiber optic tube may also be coupled within the electrode 406h in order to direct therapeutic light. In use, the RE electrode may initially apply energy to the target in order to break the skin, which envelops the needle within the target, RF energy may then be turned off and light energy is applied within the target. Alternatively the therapeutic light energy may be applied before, simultaneous, pulsed in conjunction, or pulsed separately from RF energy. A therapeutic light application in conjunction with a RF energy application is useful for example when light energy is preferred in order to treat a target, for example using blue light to treat acne. Accordingly because the needle penetrates the skin thus transmitted light energy and does not have to pass through the epidermis to reach a target beneath the epidermis, a low power light source may also be used which is preferential to minimize damage to surrounding tissue. Alternatively the electrode 406h may simply be a needle (e.g. non-RF) and the device 400 would use only a light based therapy. The light source may be used in all the examples shown and described in this disclosure.

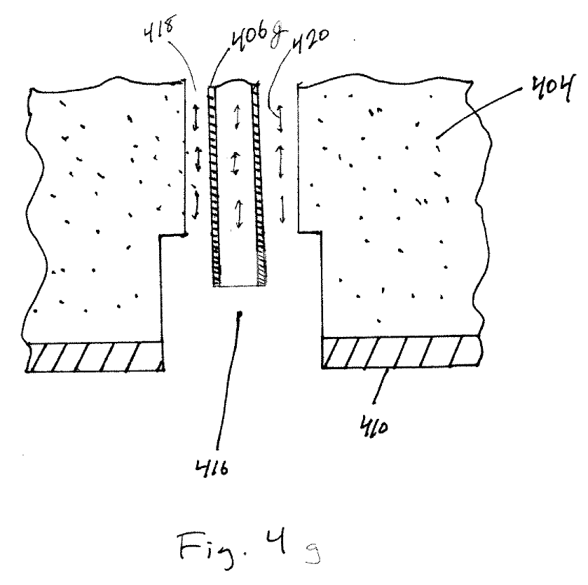

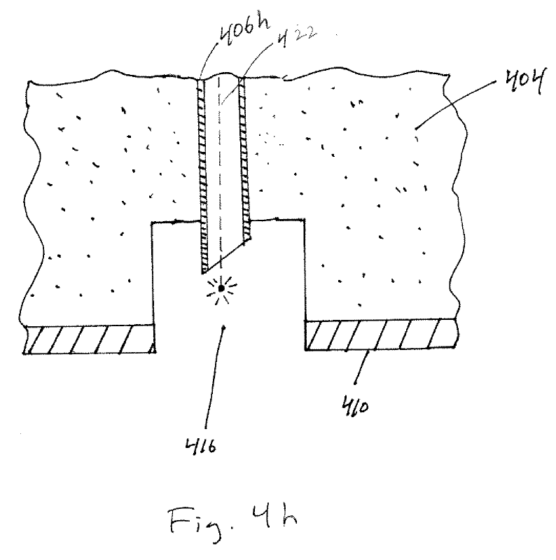

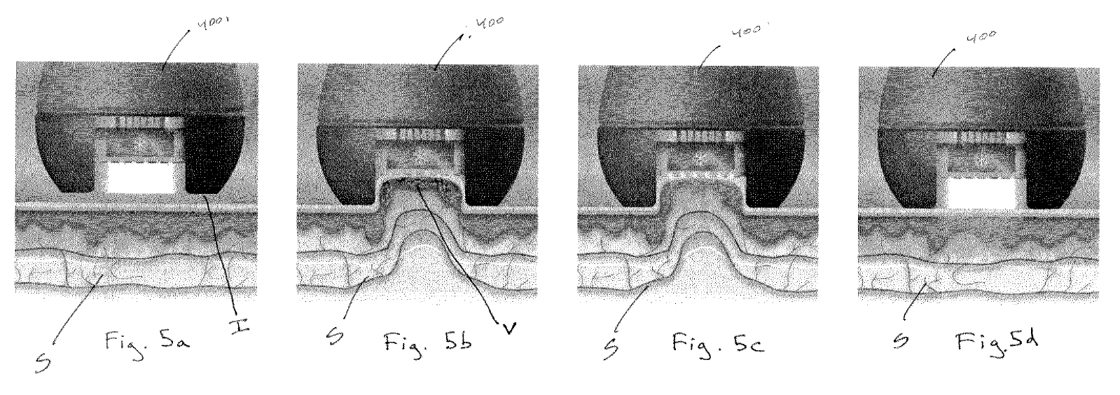

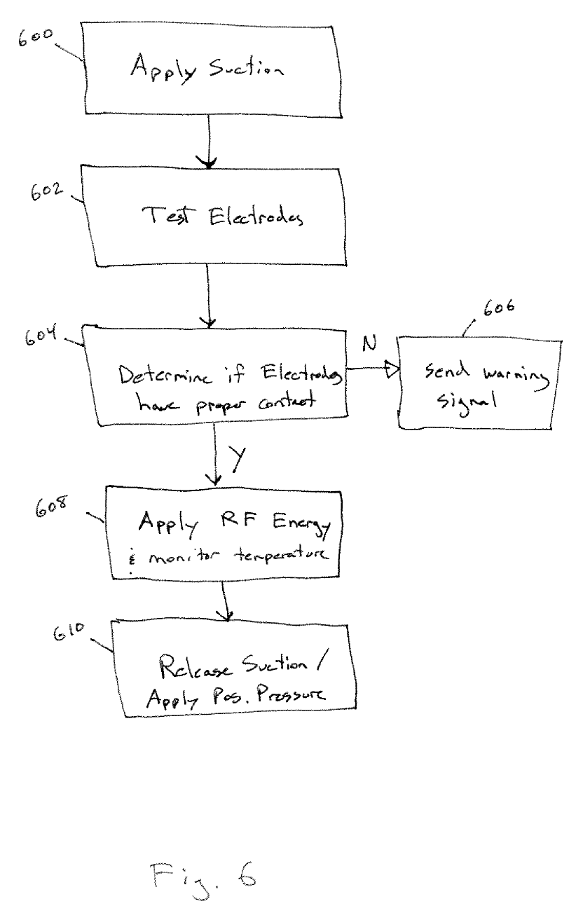

The use of one or more of the embodiments of device 400 is shown in FIGS. 5a-5d. In FIG. 5a the device 400 is placed over a portion of skin S. Positive air pressure may be applied to the portion of skin which requires an operator to apply a sufficient amount of physical force on the device 400 to form a proper seal at the device/skin interface. Negative pressure is then applied to the portion of skin to draw the portion of skin into the main chamber 414 and the electrode chambers 416 in order to define a volume of skin V for treatment, as shown in FIG. 5b. A therapeutic substance may be applied to the portion of skin S prior to applying the device to increase the electrical conductivity between the electrodes and the portion of skin S. The therapeutic substance may also or alternatively be applied after the completion of the energy application, and may provide a soothing sensation to the skin, or any substance witch will improve the clinical outcome of the treatment, such as any substance to increase the generation of collagen.

In FIG. 5c energy is applied to the portion of skin at the electrode chambers 416 by the electrodes 406. Energy may be applied by independent or incremental electrode activation, as described above. In FIG. 5d positive pressure is applied to release the portion of skin. Therapeutic substances may also be applied at this stage by the device 400.

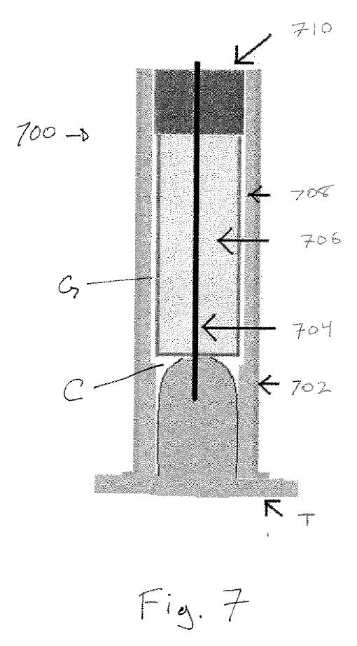

FIG. 6 shows a flow chart for the operation of the controller 102 when applying energy to a portion of skin. In module 600 the controller 102 operates the electronically controlled regulator mechanisms 110 to create negative air pressure within the chamber 416. The negative pressure in turn draws tissue into the chamber 416 to define a volume of tissue for treatment and bring the tissue into contact with the electrodes.

In module 602 the controller 102 tests the sufficiency of contact with each electrode. The controller may test the impedance level of each electrode circuit to determine if the impedance is low enough for a proper treatment. A low level RF signal or test energy may be applied to determine the impedance values. It has been determined that an impedance value in the range of 10-400 ohms would be an acceptable range of impedance to apply RF energy to tissue. The controller 102 then determines if the impedance value is appropriate to continue treatment in module 604. If the controller 102 determines that the impedance values are too highs then a warning signal may be transmitted to an operator in module 606. If the controller 102 determines that the impedance values are at an appropriate level to continue treatment then the operation may proceed to module 608.

The controller 102 may be configured allow a certain percentage of electrodes to fail the impedance test if the location of the failed electrode(s) is within a region of functioning electrodes. For example six failed electrodes spread through out the treatment zone may still offer an effective therapy; conversely six adjacent failed electrodes may not be preferable for an effective treatment. Alternatively the controller 102 may be configured to proceed with a percentage of suitable electrodes regardless of location (e.g. 100%, 90%, 60%).

In module 608 the controller applies RF energy to the tissue. It has been found that 15 watts of RF energy applied for a duration of 4 seconds is effective at treating tissue. The controller may apply energy independently or incrementally as described above. The controller may repeat the treatments until a predetermined temperature is reached within the volume of tissue. The device applying RF energy can include temperature sensors to measure the temperature rise of the skin. The RF power applied to the skin may be constant, that is the RF voltage will be constant but the current will be variable. Alternatively, the RF current will be constant while the radio frequency voltage will be allowed to change.

As shown in module 610, once the volume of skin has been treated the controller would operate the electronically controlled regulator mechanisms 110 to create positive air pressure within the chamber 416 and release the volume of skin. Alternatively suction may also be incrementally applied with each respective electrode, i.e. negative pressure would only be applied within an electrode chamber which houses an electrode which is actively treating tissue. This is particularly advantageous because it allows use of the device with a relatively low source of negative or positive pressure because the area of treatment is very small.

FIG. 7 shows a device 700 for treating a portion of skin. The device includes a return electrode 702, which also serves as the main housing for the device 700. A handle (not shown) and electrical connections (not shown) would be located on the upper portion of the device 700. The return electrode 702 may be a stainless steel tube with a flared end for contacting tissue as shown. The return electrode 702 would be insulated on the outer exterior portion of the device, using a heat shrink tubing or an insulating coating such as PTFE or polyimide. The device 700 includes a singular and centrally located electrode 704; in alternative embodiments, the device 700 may have a plurality of electrodes which are similar to electrode 704. As shown electrode 704 is a solid needle, but may incorporate other shapes as shown in FIGS. 4b-4h. If the electrode is hollow, the hollow portion may be used to deliver substances, which as an acne treatment medication. The electrode 704 may be metal, such as stainless steel. The electrode 704 may additionally be coated with a lubricous coating such as titanium nitride. The electrode 704 may transmit RF energy to the tissue T. The device includes a cold cylinder 706 and a thermoelectric cooler 710. The thermoelectric cooler 710 serves to cool the cold cylinder 706 in order to cool the tissue T. The cold cylinder 706 may be constructed from a ceramic material, or alternatively a highly thermally conductive metal such as aluminum or copper. The cold cylinder 706 would be externally insulated at the tissue T interface if constructed from a metal.

The device 700 includes an insulator 708 which lines the interior of the return electrode 702. The insulator 708 serves to insulate the interior of the return electrode 702 from the rest of the device 700, and also shapes the tissue chamber C. The insulator 708 may be constructed from insulating ceramic or polymer materials. The inner diameter of the insulator which defines the chamber C may range from 0.5 5 mm. A gap G is located in between the insulator 708 and the interior of the device 700. Positive and negative pressure may be applied through the gap G to the tissue chamber C.

Alternatively the return electrode 702 may be absent and the insulator 708 would serve as the main housing of the device 700. Thus a separate return electrode may be placed on the tissue T, or an electrode return ring would be located at the bottom of the insulator 708 at the tissue interface. The return electrode 702 may then be constructed from a transparent material, such as clear polycarbonate or glass. A transparent material would allow a user to visually align a target with the electrode 704. Alignment marks may be included on the transparent material, as well as magnification windows. The device may also include a low power laser, (e.g. non-therapeutic) which serves as a target locator. In use the low power laser would emit light onto a portion of skin as a light “dot” which a user would align with a target (e.g. hair follicle) that corresponds to a location of the electrode 704. The user would then be able to activate negative pressure and apply energy through the electrode accurately to the target. The low power laser may be used in conjunction with a transparent housing, which would allow the user to align the device with a target while the housing is against a portion of skin because the “dot” would be visible through the housing.

In use the device 700 is particularly advantageous when precision treatment of tissue areas is desired. Examples include hair removal, acne treatment, acne scar treatment, stretch mark treatment, vascular region treatment, pigmented lesion treatment, cellulite treatment, as well as general collagen growth and adipose tissue removal. The device 700 may be used in conjunction with the system as shown in FIG. 1. The device 700 may also mate with a hand held device 200 such as shown in FIG. 2.

The term functional contact includes both direct physical contact (e.g. an electrode touches the skin) and sufficient proximity (without contact) such that therapeutically sufficient RF energy can be delivered without contacting the skin.

In the foregoing specification, the invention has been described with reference to specific exemplary embodiments thereof. It will be evident that various modifications may be made thereto without departing from the broader spirit and scope of the invention as set forth in the following claims. The specification and drawings are, accordingly, to be regarded in an illustrative sense rather than a restrictive sense.

What is claimed is:

1. A medical device for treating a portion of skin, comprising:
a housing including a outer housing surface, an inner housing surface at least partially defining a main chamber for treating a volume of skin, and a sealing surface, wherein placing the sealing surface against a portion of skin forms a portion of the main chamber, the housing surface including at least one channel in fluid communication with a pressure source; and
a needle electrode coupled to the inner housing surface, wherein the electrode is recessed within the main chamber and configured to apply energy when negative pressure is applied through the at least one channel and when the needle pierces the portion of skin.

2. The medical device of claim 1 wherein the needle electrode applies RF (radio frequency) energy.

3. The medical device of claim 1 additionally comprising a controller coupled to the needle electrode and to a return electrode plate coupled to the housing surface and wherein the controller is configured to deliver the energy through the needle electrode for at least one of (a) a predetermined period of time or (b) a period of time determined based on data from a temperature sensor.

4. The medical device of claim 1 wherein the needle is hollow.

5. The medical device of claim 4 the housing is configured to apply a therapeutic substance though the hollow needle.

6. The medical device of claim 1 additionally comprising a thermoelectric cooler coupled to the housing.

7. The medical device of claim 1 wherein at least a portion of the housing is transparent.

8. The medical device of claim 6 additionally comprising a therapeutic light source coupled to the needle which is hollow.

9. A method for treating a portion of skin, comprising:
applying a device against a portion of skin, wherein the device includes a needle located in a chamber, the needle configured to apply energy to the volume of skin, wherein the needle is not in contact with the portion of skin when the device is applied without a vacuum to the skin;
applying negative pressure to the portion of skin within the device to bring a volume of skin into the chamber;
piercing the volume of skin with the needle; and
activating the device to apply an energy to the volume of skin.

10. The method of claim 9 wherein piercing occurs concurrently with activating.

11. The method of claim 9 additionally comprising aligning the needle with a target on the portion of skin.

12. The method of claim 1 wherein the target is one of a group consisting of a hair, acne, acne scar, stretch mark, vascular region, pigmented lesion, or cellulite.

13. The method of claim 9 additionally comprising applying a therapeutic substance through the needle to the volume of skin.

14. A medical device for treating a portion of skin, comprising;
a housing including a outer surface, an inner surface defining a recessed chamber, and a scaling surface for sealing the inner surface against a portion of skin and at least one passage in fluid communication with a pressure source;
a plurality of electrodes coupled to the housing, each electrode located within the recessed chamber; and
a controller coupled to the plurality of electrodes to apply energy incrementally over time through different sets of the plurality of electrodes.

15. The medical device of claim 14 wherein a spacer, which is coupled to the inner surface and is within the recessed chamber, comprises a plurality of electrode chamber surfaces, each electrode chamber surface forming an electrode chamber, each electrode chamber surface located at a distal end of each spacer passage of a plurality of spacer passages.

16. The medical device of claim 15 wherein each electrode has a distal most end which is located proximally behind a distal surface of the spacer and within the electrode chamber.

17. The medical device of claim 16 additionally comprising a thermoelectric cooler coupled to at least one of the spacer and the plurality of electrodes.

18. The medical device of claim 14 wherein each electrode is a needle configured to pierce the portion of skin.

19. The medical device of claim 14 wherein each electrode is configured to have energy applied to it incrementally with respect to other electrodes.

20. A method for treating a portion of skin, comprising:
applying a device against a portion of skin, wherein the device defines a volume of skin for treatment;
activating the device to apply a plurality of energy applications to the volume of skin, wherein the plurality of energy applications are applied by the device incrementally during a single application of the device, and wherein each respective energy application is applied to a different portion of the volume of skin.

21. The method of claim 20 wherein the device applies the plurality of RF energy applications through a plurality of electrodes.

22. The method of claim 21 wherein the electrodes are configured as needles.

23. The method of claim 20 wherein the device defines a volume of skin for treatment by applying negative pressure to the portion of skin to bring the portion of skin into the device.

24. The method of claim 20 wherein the device monitors the temperature of the volume of skin while applying energy.

25. A system for treating a portion of skin, comprising.
a controller for supplying energy to a hand held device;
a hand held device coupled to the controller; and
a tip detachably coupled to the hand held device, the tip comprising:
a housing including a outer surface, an inner surface defining a recessed chamber, and a mating surface for sealing the inner surface against a portion of skin, and a connecting surface for connecting to the hand held device;
a plurality of treatment electrodes coupled to the housing, each treatment electrode located within the recessed chamber; and;
an electrical connector coupled to the housing, the electrical connector electrically connected to the treatment electrodes.
